(19) United States
(12) Patent Application Publication
(10) Pub. No.: US 2022/0343194 A1
(43) Pub. Date: Oct. 27, 2022

(54) SYSTEM AND METHOD FOR AN ARTIFICIAL INTELLIGENCE ENGINE FORECASTING WATER QUALITY

(71) Applicant: True Elements, Inc., Naples, FL (US)

(72) Inventors: William C. LOUISELL, III, Mt. Pleasant, SC (US); David BANKSTON, Naples, FL (US)

(21) Appl. No.: 17/728,656

(22) Filed: Apr. 25, 2022

Related U.S. Application Data
(60) Provisional application No. 63/180,475, filed on Apr. 27, 2021.

(51) Int. Cl. G06N 5/04; G16Y 10/35

(52) U.S. Cl. CPC G06N 5/04; G16Y 10/35

(57) ABSTRACT

Systems, methods, and non-transitory computer-readable storage media for predicting the water quality within a geographic area based on hydrology data, contaminant data, and/or weather data using Artificial Intelligence (AI). The system can receive hydrology data for a predefined geographic region and real-time sensor data associated with water quality within the predefined geographic region. The system can then initiate a serverless AI algorithm using the hydrology data and the real-time sensor data, then receive output of the algorithm including an initial water quality score. The system can then adjust the initial water quality score based on contaminants within the predefined geographic region and transmit the resulting water quality index score to a mobile computing device.

### PRIORITY

The present disclosure claims priority to U.S. Provisional Patent Application No. 63/180,475, filed Apr. 27, 2021, the contents of which are incorporated herein in their entirety.

### BACKGROUND

### 1. Technical Field

The present disclosure relates to predicting, water quality levels, and more specifically to filtering and normalizing Artificial Intelligence results based on detected contaminants to provide an indexed water quality level.

### 2. Introduction

The health and welfare of individuals, communities, natural resources, business, government, and industry depends on sustainable water quality. The contemporary examples of human and economic devastation as a result of water quality failures are well known. There currently exists significant disparate and unstructured data on the present state of water in any given location. What has been conspicuously absent to date is any process, software, or any other viable tool capable of forecasting water conditions with scientifically reliable accuracy.

Forecasting the quality of water is a complex, multi-layered process. A technical problem of forecasting the water quality at any given location is obtaining relevant data about what is in the water sources which feed into that location, then processing that relevant data in a timely, accurate manner which can provide meaningful information to an end user. This problem is augmented by the geographic distances between sensors, differences in data collected by respective sensors, identifying the impact of various contaminants within the water supply, etc.

### SUMMARY

Additional features and advantages of the disclosure will be set forth in the description that follows, and in part will be understood from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Disclosed are systems, methods, and non-transitory computer-readable storage media which provide a technical solution to the technical problem described. A method for performing the concepts disclosed herein can include, though is not necessarily limited to: receiving, at a computer system from at least one public database, hydrology data for a predefined geographic region; receiving, at the computer system from at least one private Internet of Things (IOT) device, real-time sensor data associated with water quality within the predefined geographic region; initiating, via the computer system, execution of a Artificial Intelligence (AI) algorithm using the hydrology data and the real-time sensor data; receiving, at the computer system, output of the AI algorithm, the output comprising an initial water quality score; adjusting, via the computer system, the initial water score based on contaminants within the predefined geographic region, resulting in a water quality index score; and transmitting, via the computer system, the water quality index score to a mobile computing device.

A system for performing the concepts disclosed herein can include: at least one processor; and a non-transitory computer-readable storage medium having instructions stored which, when executed by the at least one processor, cause the at least one processor to perform operations comprising: receiving, from at least one public database, hydrology data for a predefined geographic region; receiving, from at least one private Internet of Things (IOT) device, real-time sensor data associated with water quality within the predefined geographic region; initiating execution of a Artificial Intelligence (AI) algorithm using the hydrology data and the real-time sensor data; receiving output of the AI algorithm, the output comprising an initial water quality score; adjusting the initial water score based on contaminants within the predefined geographic region, resulting in a water quality index score; and transmitting the water quality index score to a mobile computing device.

A non-transitory computer-readable storage medium configured as disclosed herein can have instructions stored which, when executed by at least one processor, cause the at least one processor to perform operations which include: receiving, from at least one public database, hydrology data for a predefined geographic region; receiving, from at least one private Internet of Things (IOT) device, real-time sensor data associated with water quality within the predefined geographic region; initiating execution of a Artificial Intelligence (AI) algorithm using the hydrology data and the real-time sensor data; receiving output of the AI algorithm, the output comprising an initial water quality score; adjusting the initial water score based on contaminants within the predefined geographic region, resulting in a water quality index score; and transmitting the water quality index score to a mobile computing device.

### BRIEF DESCRIPTION OF THE DRAWINGS

FIG. 1 illustrates an example of continuous function generation for effects of a specific contaminant;

FIG. 2 illustrates an example of mufti-dimensional effects for multiple contaminants;

FIG. 3 illustrates an example of water interconnectivity;

FIG. 4 illustrates an example hydrology map;

FIG. 5 illustrates an example of process nodes;

FIG. 6 illustrates an example water pedigree;

FIG. 7 illustrates examples of water alerts operating through a consumer app;

FIG. 8 illustrates an example data model;

FIG. 9 illustrates examples of databases used to predict water quality;

FIG. 10 illustrates an example of an A.I. engine for predicting water quality;

FIG. 11 illustrates an example of a server architecture for predicting water quality;

FIG. 12 illustrates examples of water properties at various points;

FIG. 13 illustrates an exemplary water quality platform;

FIG. 14 illustrates example screens from a water quality app;

FIG. 15 illustrates an example of contaminant lists and regulations regarding water quality;

FIG. 16 illustrates a first example of water quality indexing based on tiered contaminants;

FIG. 17 illustrates an example of water quality indexing and associated recommendations;

FIG. 18 illustrates a second example of water quality indexing based on tiered contaminants;

FIG. 19 illustrates a third example of water quality indexing based on tiered contaminants;

FIG. 20 illustrates an example of ranking health factors;

FIG. 21 illustrates an example of clustering contaminants by type;

FIG. 22 illustrates an example of ranking contaminants;

FIG. 23 illustrates an example of scoring water quality based on contaminant scores;

FIG. 24 illustrates an example of using an A.I. engine in sequence with contaminant monitoring to create a water quality index and associated suggestions;

FIG. 25 illustrates an example method embodiment; and

FIG. 26 illustrates an example computer system;

### DETAILED DESCRIPTION

Various embodiments of the disclosure are described in detail below. While specific implementations are described, it should be understood that this is done for illustration purposes only. Other components and configurations may be used without parting from the spirit and scope of the disclosure.

Disclosed herein are various configurations and embodiments of methods, systems, and non-transitory computer-readable storage mediums which utilize computationally efficient Artificial Intelligence systems to analyze water quality, resulting in predictions that can be readily used to save expensive resources and preserve the health and welfare of all end users. This results in an initial water quality score which can be effectively used and understood by persons responsible for water quality, health and safety in the government, in the private sector, as well as at a consumer level, thereby preventing hazardous water contaminant related incidents and enabling the most efficient use of human and financial resources in an environmentally and socially responsible manner.

One exemplary, non-limiting, practical application to the technical problem noted above is to receive current hydrologic event forecasts; in-situ water quality; potential for agricultural activity generated contaminant loadings; potential for storm water borne contaminant loadings; potential for industrial activity generated contaminant loadings; potential for water treatment activity generated loadings; and potential for wastewater treatment generated and/or pass through loadings to forecast changes in water quality for a specific geo-location along surface water system paths (streams, rivers, lakes, and coastal estuaries) for a specific window of time. The forecast mechanism considers such factors as: land use, soil data, seasonality, and surface water information quality (physical, chemical, biological, metallurgical, and radiological properties) flow volume, and flow velocities) provided by monitoring public & private, real-time & near real-time, sensor data, where data can be developed from authoritative public historical data bases (i.e., discharge monitoring reports) and/or adjudicated private engineering estimates (i.e., process planning and management reports). The combination of public and private data with sensor and report based data is supported through unique data normalization. The spatially and temporally structured normalized data can be input into an AI (Artificial Intelligence) engine (an algorithm), which generates forecast outputs in the form of an overall water quality level for the given location, while also identifying likely contaminant constituency and changes in levels of prevalence of the contaminants within surface water elements for the given location. The result is an accounting of the accumulation and deprecation of water quality impacting constituents resulting in referenceable ‘water pedigree’ and an indexed water quality level. Together, the water pedigree (which represents the water quality metadata) and the water quality index (which represents a numerical score) provide actionable information to stakeholders to support development, and recommendations regarding mitigation strategy prioritization, and provide factors supporting business case analysis that consider life cycle cost elements (capital investment, operating cost, maintenance & sustainment costs, salvage cost, and required return on investment).

In practice, each individual step is a technically complicated process. For example, collecting current hydrology data from public and private data services for a specific geographic area (such as a watershed) for a given geographic location requires normalization between sensor report data formats, datum references, and units of measure. The data normalization also includes spatial and temporal tagging to support down-process data operations. As an example, real-time sensor data may be obtained from disparate public and/or private databases, as well as IoT (Internet of Things) devices (such as smart valves, smart meters, smart showers, smart faucets, smart toilets, etc.). These sensors, deployed at various locations within the geographic area, often report to disparate public or private databases. Further, IoT data may or may not provide consistent data, such that the recipient of the data may need to adjust, “fill in,” or otherwise compensate for missing data points. Such normalizing or “filling in” of the data using algorithmic methods generates (from a distributed point data) a consistent data fabric such that every geographic point of interest can be queried. These methods can go beyond simply averaging neighboring data. In fact, the process is based on finding authoritative data associated with a specific location or locations that have exact or near-exact profiles as measured within the method across six property sets: climate properties, soil properties, topological properties, plant species habitation properties, animal species habitation properties, and land use properties. Each of these property sets is underpinned by measures (e.g., between four and eight) (where the measures provide specific characteristics about the respective property set/type) providing a total of 36 aspects for use in determination of ‘sameness’ or ‘difference’ for the purpose of suitability for use in creating the necessary ‘infill data.’

A high-level expression could be given as the use of information about similar rain regions, similar soil properties, and similar land use while recognizing seasonal variance in contaminants constituency, and adapting to differences in topology etc., to predict what the missing data is likely to be. In some configurations, systems configured as disclosed herein can implement a feedback system, such that the mechanisms used to make predictions of missing data can be periodically updated (e.g., machine learning), resulting in future iterations of the normalized data having refined predictions because the factors used to make the predictions have been modified. Such modifications to the predictive factors can, for example, be identified using simple regressions or higher level deep learning mechanisms for adjusting which factors most heavily influence the predicted missing data.

The AI associated with this activity can be specific to the water quality phenomena and the character of the multiple interacting factors that affect water quality at a point, at a time, under specific conditions. The systems and methods for implementing this AI, are unique for several reasons. First is the character of the underlying data which is, by its nature ‘somewhat approximate’ rather than ‘fully precise.’ For example, soil properties may be similar from point-to-point but are rarely exactly the same—differences in plant, animal, and human activity can generate differences in water quality effects from land parcel-to-land parcel, even when founded. on the same fundamental soil base. Second is the nature of interactions between the previously referenced six property sets are ‘somewhat elastic’ rather than ‘fully fixed.’ For example, biological and chemical interactions are very much influenced by temperature, humidity, moisture content, hours of daylight, etc., so it is expected that it would be extremely rare to find the exact same water quality affects in two or more apparently similar eco- and/or micro-climate systems. Third is the multitude of interaction vectors that can be present such that the influence of one particular parameter might be changed by the value of another parameter. For example, the rate of decay of an organic material, its ultimate fate, and water quality affect may be affected by changing light conditions (e.g., sunny or cloudy), as well as by changing flow conditions (e.g., the presence of a constraining wind flow opposite the current can slow surface speeds changing the effect).

Out of necessity specialization using continuous model training may be required. Systems configured as described herein can begin with the human-involved, science-based development of an initial causal map that treats each of the previously described six property sets specific phenomena as a system within which the relationships between one variable and another have multiple degrees of freedom. Degrees of freedom can be expressed as those that characterize temporal variation (delay and/or rate), those that characterize strength of relationship variation (condition-based and/or variable-to-variable interdependency-based), and/or those that characterize the impulse-response function variation (linear, non-linear, step-wise) are represented. Each of these single-property set models can be calibrated using sensor and/or IOT data. using a layered, mosaic method that puts multiple (tens, hundreds, or thousands) instances of the model to work simultaneously, with each model instance being unique in one or more degrees of freedom across all variables for which multiple degrees of freedom and multiple options within the degree of freedom are available. The training process can use an elimination or inclusion option to select which form(s) of the model perform best under different circumstances, such that a ‘family of models’ is generated for condition-based selection and use. Simultaneously, systems can build a hybrid model which combines the strong elements of each member of the family of models, resulting in a single representation which is subject. to continuous refinement.

For example, a system configured as disclosed herein can create, for a given area (e.g., zip code, city, state, province, neighborhood, flood zone, etc.), multiple models which can predict how water quality for that area will be impacted by various factors (such as the interaction factors discussed above). For example, the system could have a first model to identify water quality conditions when rain levels are below a first threshold amount within a two week period, a second model to identify water quality conditions above the first threshold amount and below a second threshold amount during the two week period, and a third model to identify water quality conditions above the second threshold amount. Please note that the two week period is exemplary the time period can vary to any predetermined amount as needed. In addition, the number of models, and the predetermined thresholds used to select those models, and can be any number required by the system. If the one or more variables used to select the models are not time related, the thresholds associated with selecting particular models can be predetermined amounts of those variables. When data is recorded by sensors or received from those sensors, the system can then select the appropriate model based on how the sensor data compares to the predetermined thresholds. While in this example three different models for a specific condition (rain level) are available (which are selected based on two thresholds), in practice there can be many different models associated with different ranges and thresholds of many different variables (such as rain level, distance from an industrial site, type of fertilizers used, UV/daylight exposure, etc.). In such cases, the system may identify multiple models which are appropriate. In such cases, the system may (1) execute water quality predictions using all of the models, then combine the results using averaging or by weighting the models based on a level of predicted relevance (where the system predicts relevance based on how close the sensor data, aligns with a given model); or (2) by combining the models before execution (e.g., combining the weights of variables, particular sub-routines/analyses), where the combination is again based on predicted relevance.

On the next level, the system-of-systems level, training can be conducted in the same manner on a larger scale. Nominations of single property set models (sub-models) can be integrated into the larger causal map. Like in the single system process described above, the multi-system model can be run as a layered mosaic underpinned by a record keeping function that manages the continuous selection and condition-based applicability characterization process, thereby providing an ‘on-demand’ provision of the most appropriate model for any one of the nearly finite computational elements catalogued within the system.

The A.I. engine can be it on a cloud computing system such as AMAZON WEB SERVICES (AWS), allowing the system to only use the computational resources required at a given moment. That is, such configurations can request and use additional computing resources when additional computing is required, and relinquish those computing resources when no longer needed, such that systems configured as disclosed herein can save energy by only using computing resources When required and results in a more computationally efficient system for predicting water quality than systems which have dedicated servers. The A.I. engine can also have individual computing sections/modules which individually make predictions, the outputs of which can then be combined together to make predictions for a given location. In some configurations, these computing sections/modules can be distinct computing systems, where each module has one or more module-specific processors used to perform the computations specific to the computer-readable code for that module. Such a configuration could, for example, be implemented where each module is executed on a distinct server or computing platform. In other configurations, a single computer system can execute two or more of the respective modules sequentially or in parallel. For example, a single server could be performing all of the computations of the disclosed modules, where the specific code/module executed at a given time can vary according to operational needs.

The A.I. engine, in general, receives the data stored in various databases and makes predictions from that data. For example, the A.I. engine can receive weather or other meteorological forecast data. The weather forecasts can be combined with hydrology data to make water runoff and stream flow predictions for the given area. Using the water runoff and streamflow predictions, as well as information about What contaminant load to expect within areas effected by the hydrologic event, the A.I. engine can predict how contaminants (such as chemicals and/or pollutants) will travel and spread. Based on previous sensor data and these predictions, the A.I. engine can generate a predicted water level quality on a contaminant-by-contaminant basis. This contaminant-by-contaminant basis uses the persistence and transportation characteristics of each contaminant to determine the forecast levels of each contaminant at any specific, point travel, thereby enabling a contaminant-by-contaminant forecast at any point in space and time. The forecast data can then be used to create reports for various users, such as environmental planner, agricultural manager, industrial manager, facility/campus manager, water system manager, wastewater system manager, citizen scientist, and/or individual consumer. Water quality forecast reports can include a forecaster water quality index, summary information, and detailed reports including historical comparisons.

One example module is the “Storm Effects” module, which can make weather, and specifically precipitation (rain and snow), forecasts for a given Area Of interest (AOI), The storm effects module can receive data from public/private databases regarding current water levels, current storm activity, weather patterns, seasonal data, etc. In some configurations, the system can also download weather predictions, whereas in other configurations the system can use previously downloaded or generated weather prediction models, and use those models to generate custom weather predictions. The weather predictions can include an amount of anticipated rainfall within the AOI.

Another example module is the “Hydrology” module, which can receive data from the storm effects module regarding predicted weather/rainfall in a given area, then use hydrology i.e. storm intensity, land cover, land use, and topography data to predict water runoff in terms of expected quantity, peaking profile, elevated flow duration, and time of concentration. In some configurations, this can use known hydrologic data, topographic data, location of key riverine system nodes, transport and storage characteristics of those nodes, distances between nodes, and the influence of significant man-made diversion/storage/flow management systems such as dams and overflow channels to predict the effects of the anticipated rainfall on a downstream area.

Another module which can be present within the A.I. engine is a “Sector” module. The sector module can generate contaminant loads for placement into the hydrologic flow system. Loads can be generated on a sector-by-sector basis for contaminant generation activities attributable to agriculture, stormwater management, industry, water treatment, and wastewater treatment activities. Each of these five sectors and, in-turn, their subsectors and individual member activities, can be modeled based on historical data as to their operating cycles that define contaminant-by-contaminant load generating impulses and their relationship to meteorological and/or operational cycles. Example realization cycles can be defined by factors including include hour, day, week, month, and/or seasons of the year. For example, previously collected data from sensors may indicate a higher/lower level of a given contaminant at specific times of day, days of the week, etc. Likewise there may be information stored in a database indicating frequency of use of various pesticides or other contaminants on a seasonal basis. The sector module can output a list of the contaminants which should be monitored within a given cycle.

The system can also contain an “Overland and Riverine Transport Module, which can use the data from the hydrology module, the sector module, and/or the storm effects module to predict the transportation, diffusion, and/or spread of contaminants within a specified period of time. To do so, the module can access one or more databases which identify geographic sources for various contaminants identified by the sector module, and properties associated with the dispersion of those contaminants (such as solubility, weight, etc.). Further, beyond the constituency, diffusion, and persistence aspects of transport, the Overland and Riverine Transport module can use the hydrologic predictions (in terms of runoff quantity) in combination with the localized, sensor-reported, streamflow baseline velocity data and the localized slope values generated using the hydrographic and topology data to forecast changes in flow velocity such that the downstream forecasts reflect flow changes associated with rainfall patterns (historical & forecast). The system can then provide predictions of contamination transport as output. For example, the overland and riverine transport module could receive data from the hydrology module indicating no additional rainfall is expected in the next three days for a given area. Using that data, the overland and riverine module can access a database identifying “normal” or standard contamination sources, and determine (based on the current water levels) how contaminants from those contamination sources will likely spread over the next three days. in another example, the hydrology module could indicate approximately 2 inches of rain (5.08 cm) over the next three days for the same given area. In this case, the system will not only identify contaminants from the standard contamination sources, but also access databases identifying contaminants which may spread from the rainfall, such as agricultural contaminants fertilizer runoff, anaerobic lagoons, holding ponds, etc.)) and/or vehicular pollution oil, rubber, and other road waste). In this case, the overland and riverine module would use the hydrology data, the standard contaminant data, and the additional, rainfall specific contaminant data to predict how contaminants will travel and where they will be deposited at any given point in time.

In some configurations, the A.I. engine can contain a “Predictive” module, which executes the indexing operations disclosed herein. For example, the predictive module can use the calculated predictions regarding contaminant transportation and spread to identify, for a given location, the predicted water quality level for that location.

The A.I. engine can also have a “Learning Module,” which can use contaminant sensor data to verify predicted contaminant amounts. When the predicted contaminant amounts do not match the actual contaminant amounts detected by the sensors, the learning module can identify the nature of the variation (temporal error, amplitude error, massing error, and/or duration error) to modify the algorithm(s) or algorithm element(s) across the entire system i.e. storm effects module, hydrology module, sector module, overland & riverine transport module, and/or the predictive module, to improve future predictions. In doing so, the learning module can independently generate modification candidates, back-test its various modification strategies, and then adjust from the trial results the modification strategy to determine the optimum change using a genetic algorithm approach to identify which algorithm(s), algorithm elements(s) and what coefficient and/or exponent values produce the best results. The Learning Module can publish the results across the system and monitor for the en tire learning cycle (defined as period between machine and/or analyst modification) in parallel with the base value generated solution, the published genetically generated modification solution, and the modification top five contenders to continue the deep learning cycle.

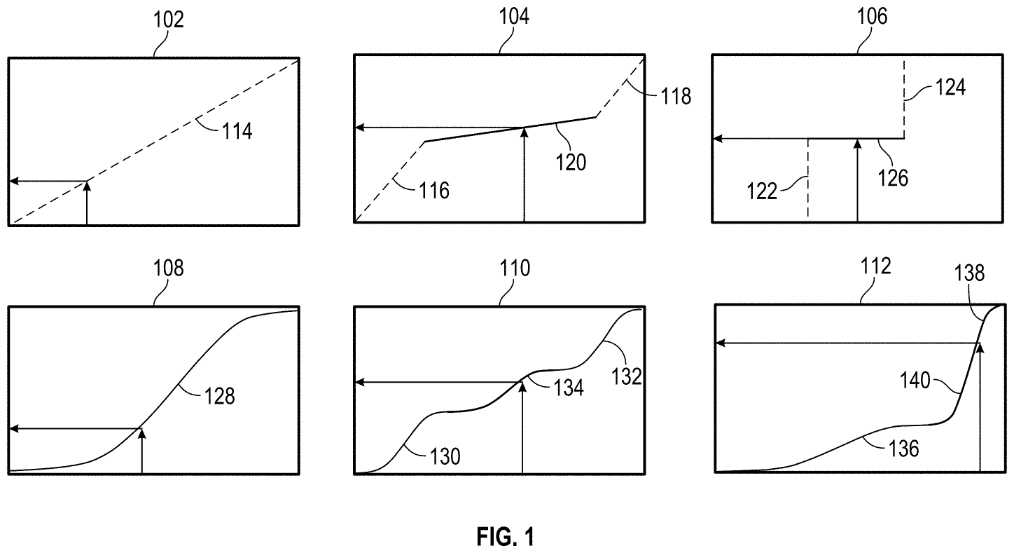

Core to the indexing system is the ability to automatically generate a continuous mathematical function that relates a level of concentration of a given contaminant, or a combination of contaminants, with a level of significance, illustrated in FIG. 1. The level of significance can, for example, indicate how much harm may be conveyed to an individual in contact with the respective contaminant. In its base form the function can be displayed on a cartesian plane such that for a given concentration (x-axis), a given level of significance (y-axis) can be found. In the case where two points are discovered via reference to authoritative data the EPA, Industry Publications, Research Publications, Manufacturer's Material Safety Data Sheet, etc.) a line function can be formed. As more points of concentration to significance relationships are discovered, the function shape may change to conform to provide an x-to-y relationship that reflects the evolving data. In this case, introduction of new data in a specific dimension, the AI engine will reform the function shape to reflect the data revealed changes to the cartesian relationship. The level of significance for any given contaminant, or combination of contaminants may be multi-dimensional and/or multi-tiered. The significance may, for example, be a measure of toxicity in one or more dimensions associated with type of contact, such as exposure by mouth, exposure by inhalation, exposure by skin contact, or any defined combination.

Further, the AI engine can track the level of severity for a given chemical interaction by level of harm indicated by an authoritative source. For example, if the system obtains information indicating different levels of harm caused by different levels of exposure, the AI engine can generate a continuous mathematical function which emulates those levels. For example, as illustrated in the top left graph 102 of FIG. 1, if the system were given two data points, the resulting mathematical function may be a linear function with a linear significance line 114, indicating that the potential results for exposure to the contaminant increase linear based on exposure. A system configured as disclosed here can simultaneously, for a contaminant of concern, track the impact of a particular concentration on at least three levels—by a single dimension (type of contact), by multiple dimensions, and on multiple tiers (where the type of harm changes when the level of significance and/or the exposure to a contaminant increases to a predetermined level).

When additional data points are retrieved (via download from a website, scraping data from a website, or other means), the system can update the previous mathematical functions to account for the updated data. In the top middle graph 104, the system updates the equation with new data points 120, such that the lower 116 and upper 118 points of exposure are updated to conform with previous data.

In another graph 106 is illustrated an example of a step-function response to exposure to a contaminant. In this example, any exposure to this contaminant will result in an immediate result (illustrated by the vertical line 122). Then there is a portion where additional data 126 is showing that additional exposure to a particular contaminant does not result in additional harm to a point. Once that point is reached, the subsequent exposure again results in immediate results, illustrated by the second vertical line 124.

In some instances, the resulting mathematical functions may be non-linear, as illustrated in the three bottom examples 108, 110, 112. As illustrated in 108, the contaminant significance may be curvilinear 128, with portions of the resulting function having sections which are essentially linear, and other sections which are curved. Likewise, in example 110, there may be portions which are known 134, and other sections 130, 132 where the system uses the equations of the known section 134 to form the overall equation. In yet another example 112, the system may gather known information 136, 138, but be missing a portion of data which implies a large increase 140 in the significance of impact. In this case the AI engine can form the missing portion 140, resulting in a continuous equation taking account of all known data.

To constantly update the data, the AI engine continuously obtains evolving data from databases and/or web scraping websites. With that data, the AI engine can assign the dimension or dimensions and tier or tiers to a respective contaminant, such that the equations relating concentration to significance can be continuously and simultaneously adjusted across each dimension and/or tier simultaneously.

To support computational efficiency, the applicable function for a specific contaminant can be foamed on the fly to form a single base equation which responds in terms of shape to a contaminant, a type of interaction (dimension), and/or a tier specific vector (type of harm) stored as a single-, double-, triple-, or quad octet in which each position can be filled with an integer value between 0 and 9.

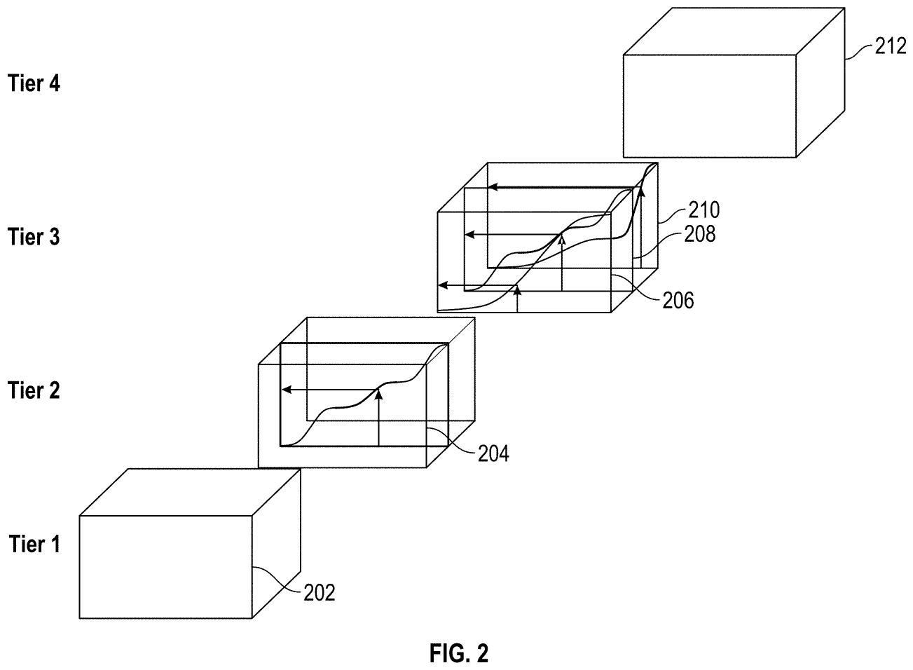

As illustrated in FIG. 2, in the case of discovery of data indicating that the contaminant of concern has impacts on multiple dimensions of significance, the AI engine can generate a cartesian relationship in each discovered dimension such that the individual and or combinatorial effects on human health and life safety can be individually tracked and simultaneously generated and delivered to the final indexing algorithm as an input. For example, in Tier 1 202, there may be no known impacts due to contact with a contaminant. In Tier 2, we see a single known type of health impact (and its equation 204) due to contact with a contaminant. In Tier 3, multiple equations/graphs exist 206, 208, 210, providing information regarding health impacts for different types of interactions. Tier 4 may represent another level of impact, where any contact 212 with the contaminant would result in serious harm to the individual.

Identifying future contaminant levels within the water supply (and their impact on water quality) for a given location can, in some configurations, be part of the A.I. engine. In other configurations, identifying the presence and impact of contaminants can be a distinct, separate process. The identification of contaminant levels can, for example, involve the use of historical data; known activities and locations of agricultural areas including crop cycles and associated nitrification, herbicide treatments, and pesticide treatments; stormwater outfalls and associated seasonal contaminant constituency: industrial sites and associated contaminant constituency, water treatment plants and associated practices, wastewater treatment plants, and associated practices, etc.; hydrology within the watershed; current and/or predicted rainfall; seasonal variance data; use triangulation and/or time-series generation methods; and/or other data descriptive of the constituency, toxicity, and volume of discharges. Collection of such data makes use of public/private databases, public/private sensors, public/private IOT devices, and public/private hydrographic and hydrologic data described above.

The system can use the identified contaminants loads to establish the initial water quality via the A.I. engine, resulting in an indexed water quality level. In practice, the system can identify the type and/or level of harm which can be caused by each contaminant, and reduce the overall water rating based on the amounts of detected contaminants coupled with their respective type/level of harm. The system can use sources such as EPA (Environmental Protection Agency) databases, industry publications, research publications, manufacturer's Material Safety Data Sheet (MSDS), etc. The result is an indexed water level quality readily understood by expert and non-experts in water quality.

In some configurations, based on the types of contaminants and/or the indexed water quality level, the system can also output recommendations regarding operational management strategies and/or investment in mitigation technologies. As an example of an operational management strategy, the system can use the forecast surface and/or groundwater water quality conditions to suggest that discharges from certain process should be delayed for a specific time such that the impact of the discharge on the receiving waters will minimize adverse downstream impact. As an example of a technology recommendation, the system can support, using private and/or public data, a recommendation of pre-treatment technologies and their associated implementation processes that will generate environmental compliance given the specific contaminants and/or operating characteristics of a business, facility, and/or campus. In this configuration, the user can define compliance requirements, operating constraints, capital investment constraints, and/or operations and maintenance cost constraints. In one such use case, for a specific user, the system can access public and/or private data on the utility and performance of filters is proper for a found contaminant (or combination of contaminants), identify the inventory level of the vendor, and make a recommendation to the user that they purchase the found filter to improve water quality given the specific contaminant constituency identified within the user's water supply. In addition (or alternatively), the system can output the indexed water quality level itself to a user. For example, if the index extended from a “100” for perfectly pure water to “0” for completely contaminated water, the system can output the indexed score to the user, with or without explanation for the various contaminants found in the water or other reasoning for the score.

Systems configured as disclosed herein retrieve and process information in ways impossible for human beings. For example, retrieval of data from sensors scattered over a geographic area, accessing disbursed databases, and performing computations using a serverless computing system all require access to a computer network, such as the Internet, comprising tangible servers and other data storage devices. In addition to the data collection process requiring tangible tools for implementation, systems configured as disclosed herein to normalize data, assign temporal and spatial tags, and assign data instructions as to the joining vectors with other data elements and to routing instructions to destination applications within the A.I. engine suite. To improve data analysis and processing of the collected information a feedback loop is employed to iteratively improve the predictions made with respect to realized storm effects, realized contaminant loading, realized contaminant transport, realized time of arrival, realized duration and intensity of impact, etc.; while constantly reducing the energy and computational expenditure by using serverless instances to execute on the data.

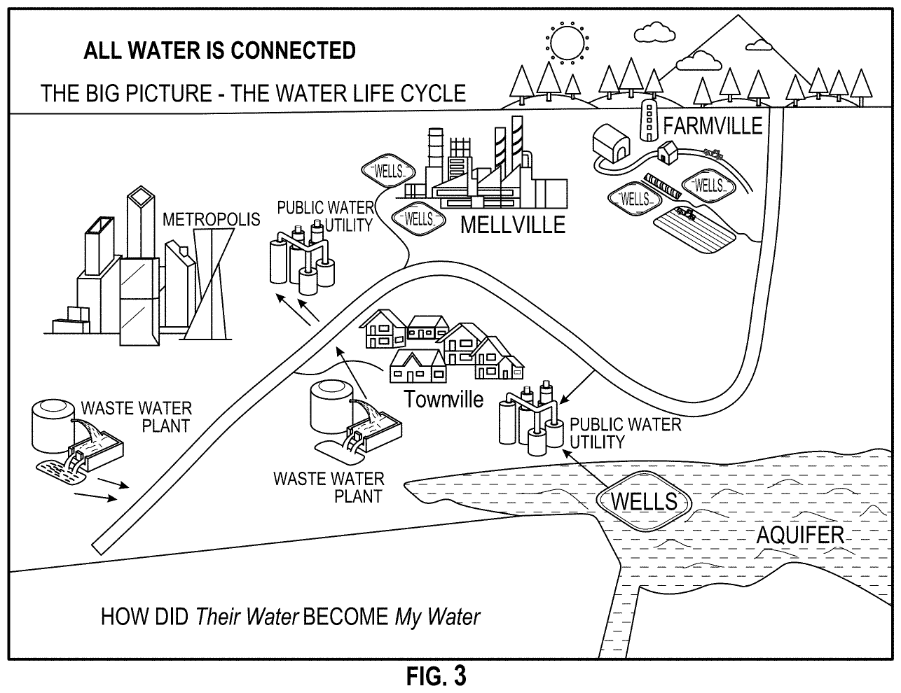

FIG. 3 illustrates an example of water interconnectivity. As illustrated, water flows from mountains and may pass by farms, to industrial centers, suburbs, waste water treatment plants, and major cities. The wastewater output by one city or industry can influence the source water for another, such that how respective entities receive, treat, and otherwise use their water can be of great concern.

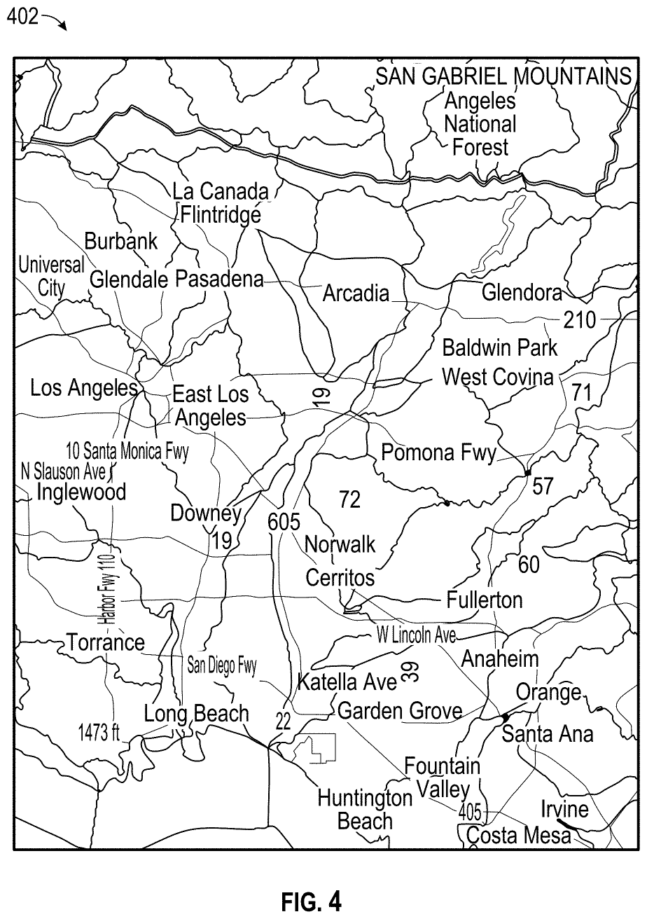

FIG. 4 illustrates an example hydrology map. A hydrology map, such as the one illustrated, identifies how water flows within a geographic area, and can include geographic coordinates (such as latitude and longitude), as well as water flow direction in cardinal the water is flowing South), bearing, and/or azimuth formats. The hydrology map may also include information regarding the elevation of respective points and/or the slope of the topography associated with each point, such that predictions of how the water would flow and/or how fast the water would flow can be made.

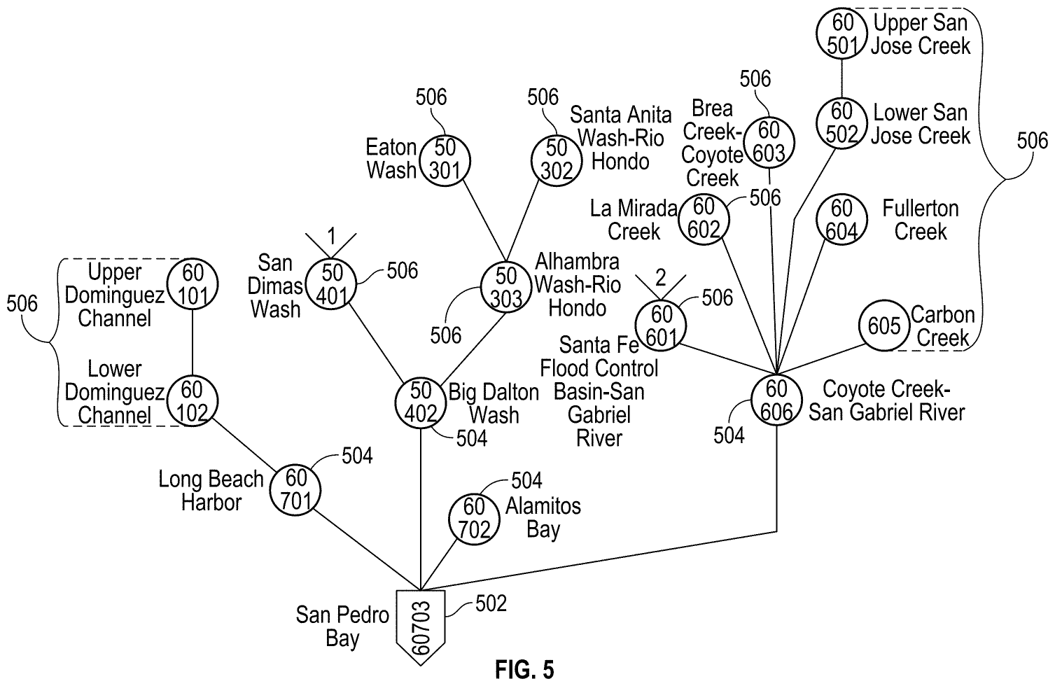

FIG. 5 illustrates an example of process nodes 502, 504, 506. The process nodes illustrate how the various portions of a hydrology map combine together, with each section of a hydrology map corresponding to a respective node within a tree. As the water moves toward the ocean 502, the process nodes combine together, allowing a visualization of how the respective portions of the hydrology map combine together. As illustrated, some nodes 504 directly feed into the ocean 502, whereas other nodes 506 combine with other nodes prior to reaching the ocean 502.

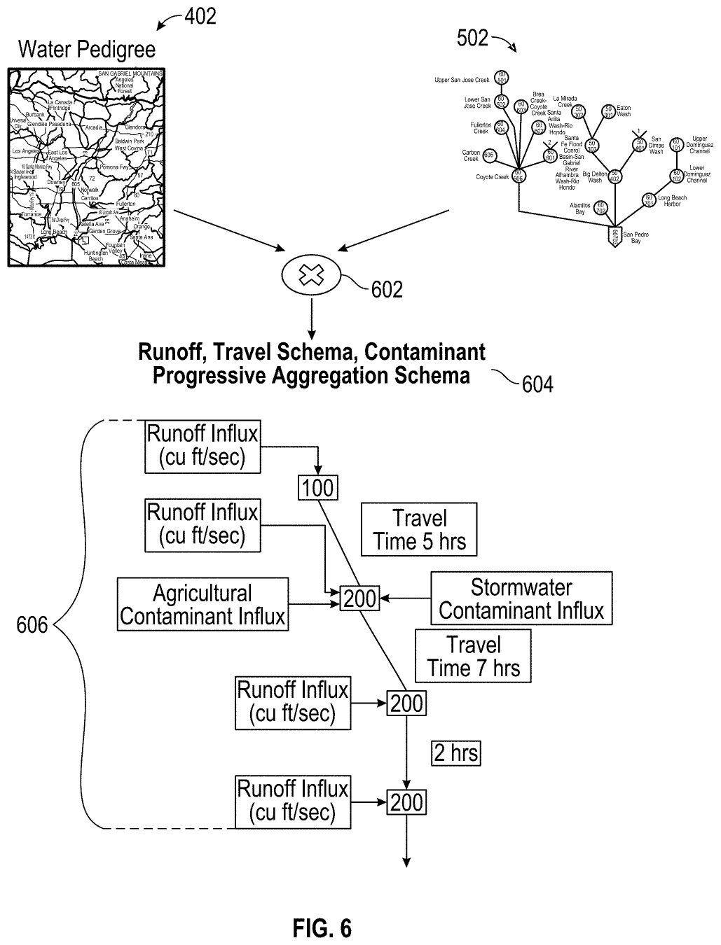

FIG. 6 illustrates an example water pedigree, formed by combining 602 the hydrology map 402 information with the process nodes 502. The combined pedigree can be used to calculate runoff, travel schema, and/or the aggregation of contaminants 604. Using a formed water pedigree, known precipitation levels, seasonal information, etc., the system can make predictions 606 about how what contaminants will be in the water and how fast those contaminants will arrive at a downstream location.

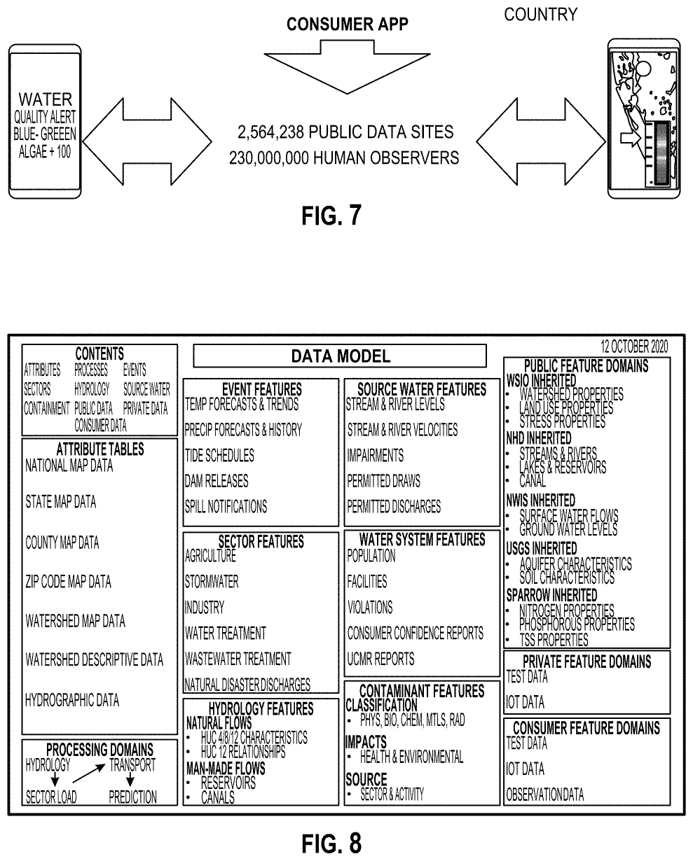

FIG. 7 illustrates examples of water alerts operating through a consumer app, which can he deployed on a smartphone, tablet, desktop computer, and/or any other computing system. As illustrated, the consumer app can interact with human observers and/or public data sites to provide water quality index scores to users. On the left is illustrated a water quality alert where the score is a “blue-green,” and the app also provides a side effect of the water quality, “Algae+100.” On the right is illustrated the spectrum of possible water quality levels and the current predicted level, with a map showing a pin of where the water quality is being predicted.

FIG. 8 illustrates an example data model. This example model provides overview of all of the different data types and data sources which can be used to execute the disclosed algorithms and make the disclosed predictions. As illustrated, some of these resources can be stored in public databases, others private databases; some of the resources can be created and stored by a system configured to generate and produce such resources, other resources can be created, stored, and accessed remotely via the system.

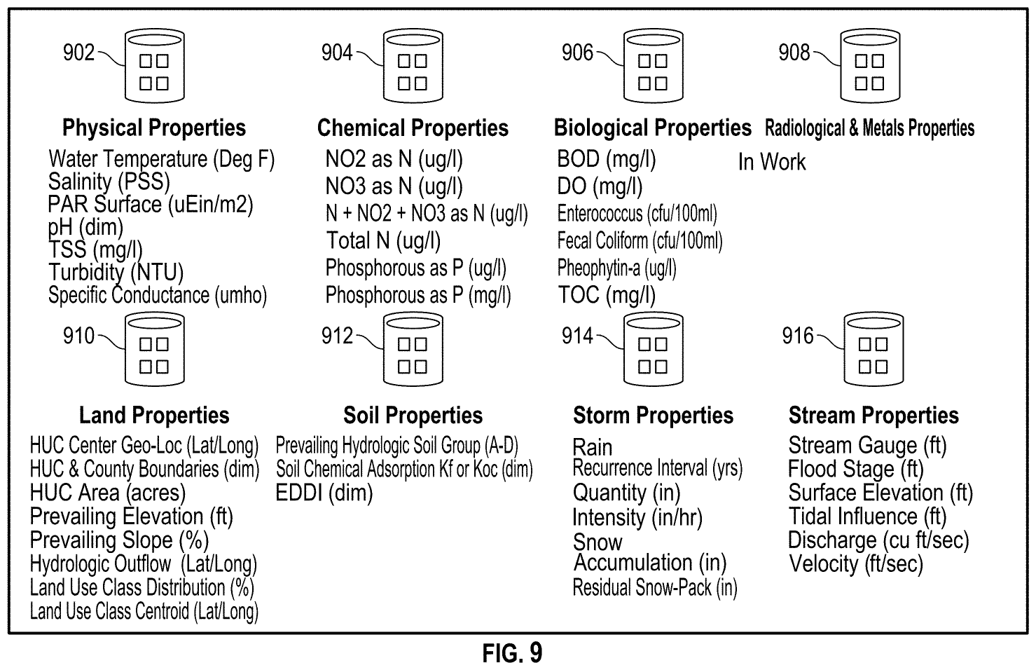

FIG. 9 illustrates examples of databases used to predict water quality. For example, at any given location, the properties which may be used to predict water quality may include physical properties 902, chemical properties 904, biological properties 906, radiological properties 908, metallic properties, land properties 910, soil properties 912, storm properties 914, and/or stream properties 916. In some configurations only some of these databases may be used, whereas in other configurations other databases may be used.

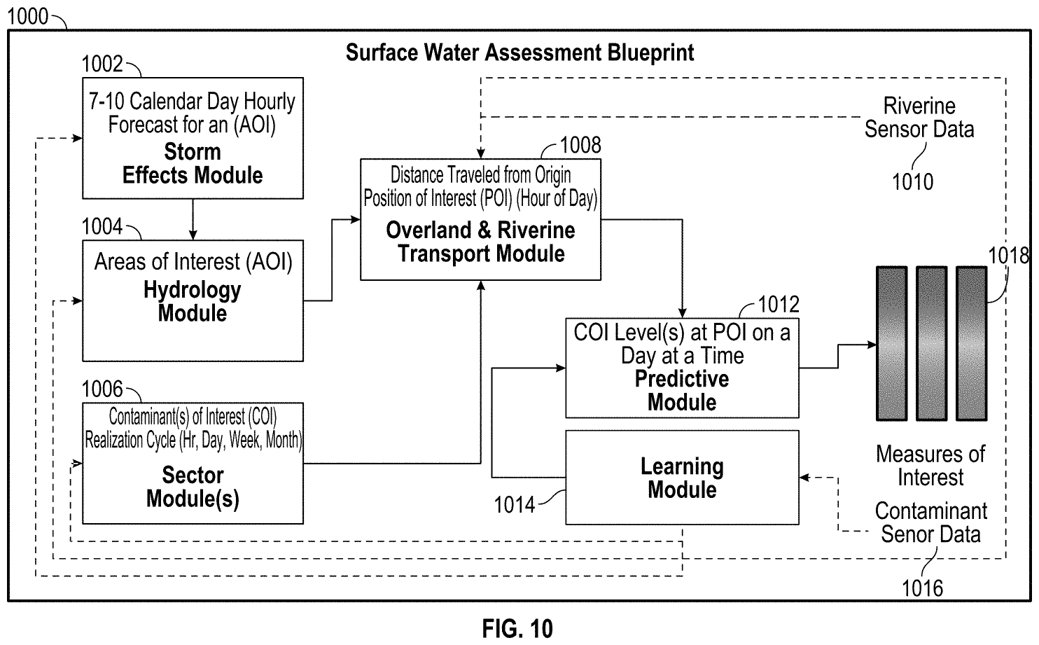

FIG. 10 illustrates an example of an A.I. engine 1000 for predicting water quality. The illustrated A.I. engine 100 has multiple exemplary modules (a “Storm Effects” module 1002 which can access hourly weather forecasts; a “Hydrology” module 1004 which can access hydrology databases; “Sector” module(s) 1006 which can identify potential contaminants of interest (COI) which may be present in a given geographic area based on hour, day, week, or month; an “Overland & Riverine Transport” module 1008, which can use sensor data 1010 to calculate the distance traveled from an origin Position of Interest (POI) based on hour of day; a “Predictive” module 1012 which can calculate water quality based on contaminant levels and the output of the Overland and Riverine Transport module 1008; and a “Learning” module 1014 which can use feedback mechanisms to modify the algorithms of the Predictive module 1012 and/or other modules within the A.I. engine, and can further rely on contaminant sensor data 1016. The final output of the A.I. engine 1000 can be a measure of interest 1018, also known as a water quality index score.

The illustrated A.I. engine 1000 can be implemented using a computer system, such as a server, or a serverless computing system. The respective modules illustrated can be executed by distinct computing systems, or can be a single computing system executing distinct portions of a computer algorithm/code. In some cases, the distinct modules can be operated in parallel, using multi-core processors, distinct servers, etc.

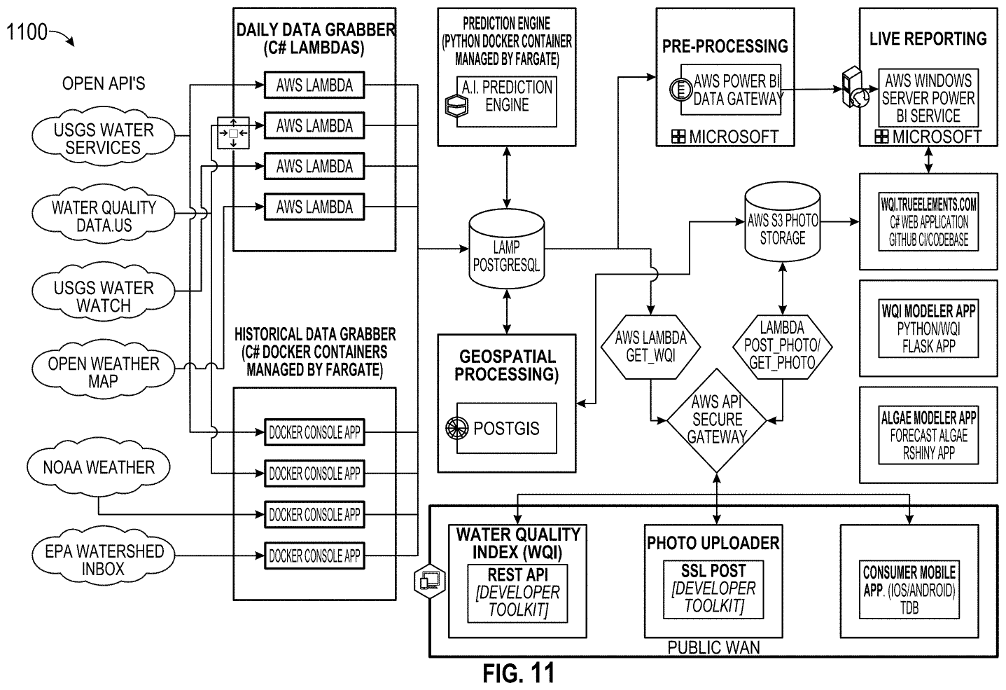

FIG. 11 illustrates an example of a server architecture for predicting water quality. In this example, the system accesses public databases using open APIs (Application Programming Interface). This data is gathered using functions such AWS Lambda or other data grabber technology, then aggregated into a database, such as a “LAMP PostgreSQL” database. The database can also include Geospatial Processed data, as needed. In some configurations, the aggregated data can be normalized, averaged, or otherwise prepared to be stored in the database. Such normalizing or “filling in” of the data can go beyond simply averaging neighboring data, and can include information about known agricultural runoff (and/or information about other pollutants or contaminants), slope/elevation data, weather predictions, use of historic data, etc., to predict what the missing data is likely to be.

The Pre-processing of the data prior to the live reporting involves generation of use case centric data that is made available to support visualizations and reports. Use cases provide a means to simplify the data presentation environment for ease of user interaction. The data generated in the extract-transform-load process is stratified according to multi-tier scoring/decomposition structure, where the overall environmental data is easily broken down into a few variables for user understanding. For example, in a four tier decomposition structure, the variables may be—overall status (color code and numerical value), access to primary supporting data (color code and numerical values), access to secondary supporting data (color code and numerical values), and underlying metadata of importance, i.e. base data that drives the upper tiers of the data presentation structure

Geospatial and temporal tagging can be used within the operations. All data can be tagged with latitude/longitude and time of origin. Such tagging supports the operations related to identifying sequences of contaminant loads for use in the water quality forecast use case in the form of the water pedigree. Geospatial and temporal tags can be generated using data headers, e.g. headers associated with digital imagery, point-of-discharge reports, and/or sensor inputs. In cases where header data is not available, the data is tagged as ‘area’ data and ‘temporal window’ data. Area and temporal window data can be used in making initial estimates of contaminant loads from agriculture and stormwater runoff. Contaminant loads with area and/or temporal window taus can be refined in terms of space and time at the first point of suitable sensor availability via the in-line application of the previously described learning algorithm.

The water quality forecasting application operates on an hourly cycle for the first 72 hours of the forecast horizon and then reverts to a six-hour cycle out to 10 days. Beyond 10 days, the operation reverts to a 24-hr cycle through the 21-day meteorological forecast horizon. Historical data can be used to generate the range of possible futures, in applicable use cases, for periods out to 1-year.

From that database a number of distinct applications can be executed in parallel, such as an A.I. prediction engine and/or Live Reporting with associated pre-processing. The results of the various prediction algorithms and live reporting algorithms can be output as a Water Quality Index to apps, models, etc. Users can then access the predictions remotely across a network via terminal computing devices such as smartphones, tablets, or other devices.

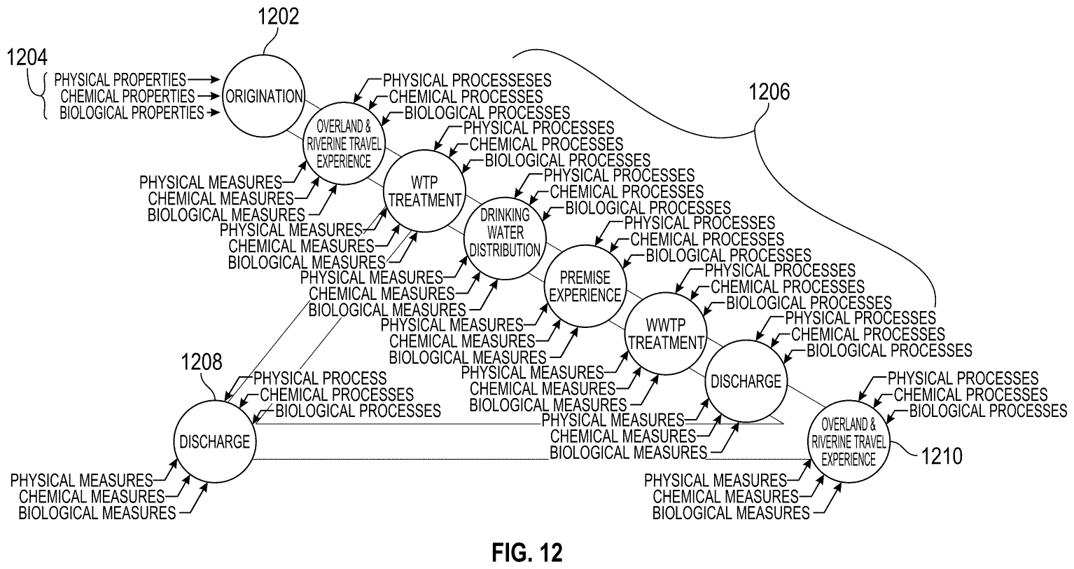

FIG. 12 illustrates examples of water properties at various points. The large circles represent the stages of water, and read: Origination 1202, Overland & Riverine Travel Experience, WTP (Water Treatment Plant) Treatment, Drinking Water Distribution, Premise Experience, WWTP (Waste Water Treatment Plant) Treatment (collectively 1206), Discharge, 1208, and Overland & Riverine Travel Experience 1210. For each circle, there can be (as illustrated) six factors: physical, chemical, and biological measures 1204 (illustrated on the left of each circle); and physical, chemical, and biological processes (illustrated on the right of each circle). These measures and processes illustrate how the system analyzes the water quality at a given location based on the water's previous stages and the inputs to those respective stages. Moreover, this figure illustrates that as water progresses further from source or origination, the number of factors and processes in determining the water quality become larger and more complex.

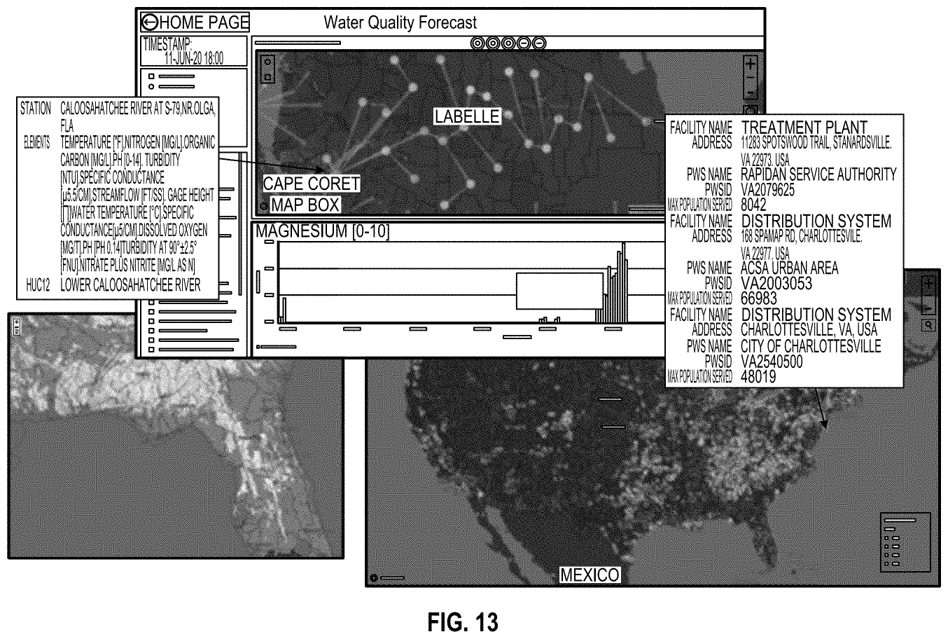

FIG. 13 illustrates an exemplary water quality platform. This is the type of data which could be communicated to an end user, with the ability to show a current water quality level for any geographic location or area, the data sources accessed to determine a given location's water quality, contaminants located or predicted at a given location, etc.

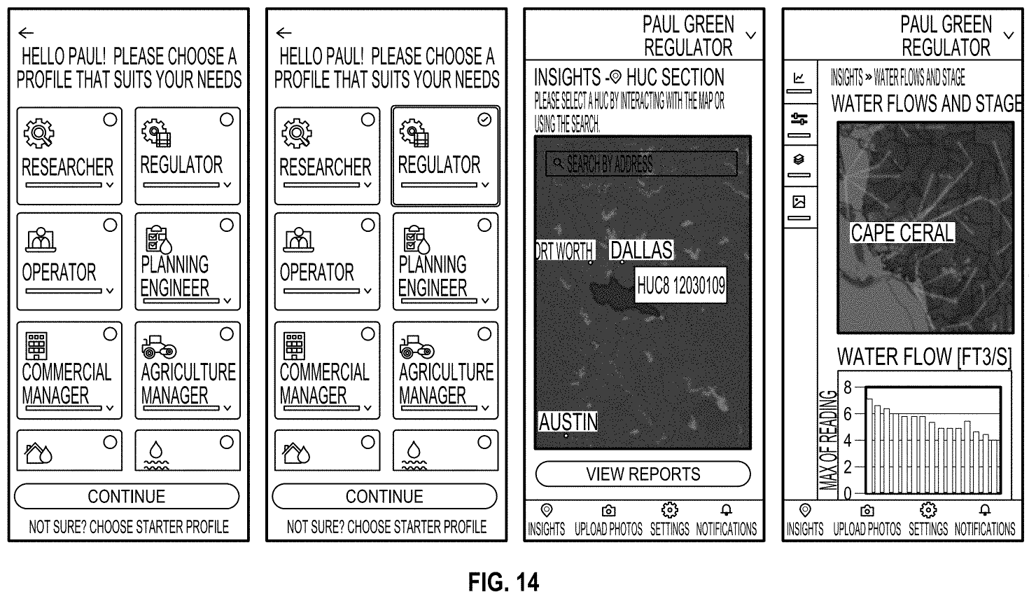

FIG. 14 illustrates example screens from a water quality app. As illustrated, the app can provide different content based on the profile of the user, and can show water flows, photos, water quality for a given location, and/or other data calculated or retrieved by the system.

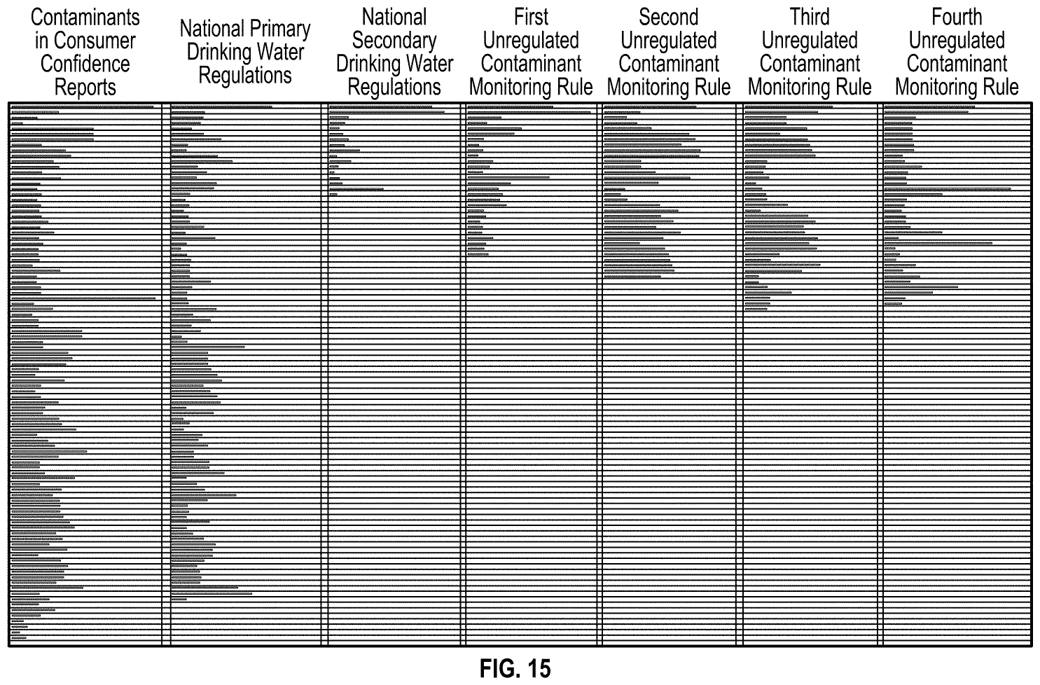

FIG. 15 illustrates an example of contaminant lists and regulations regarding water quality. Such data can be accessed by the system from databases, with each list containing names of contaminants according to various rules, regulations, and/or reports. For example, the system can retrieve a list of contaminants in a consumer confidence report, the list of contaminants according to one or more national drinking water regulations, and one or more lists of unregulated contaminants. In some configurations, the system can also retrieve lists of contaminants identified or tracked by certain states, provinces, counties, and/or other localities. These lists of contaminants can be used in determining the water quality level by the A.I. engine.

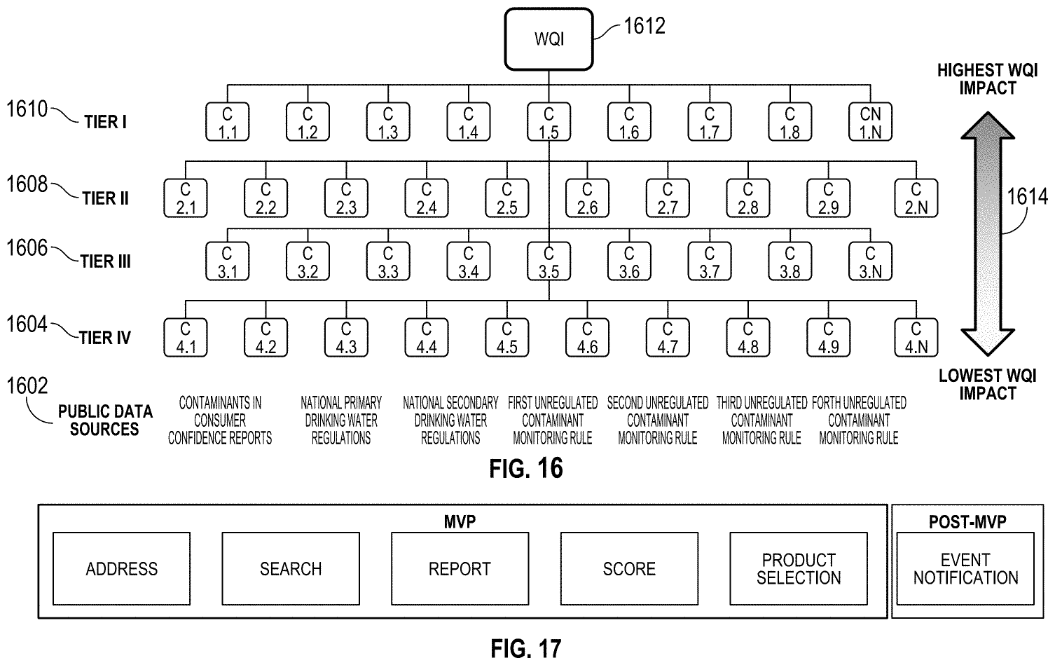

FIG. 16 illustrates a first example of water quality indexing based on tiered contaminants. In this example, the list of contaminants is tiered according to impact, with contaminants having a higher impact on water quality listed as “Tier I” 1610, and contaminants having less impact on water quality listed as tiers II-IV 1608, 1606, 1604. In other configurations additional tiers may be present. As described above, the Lists of contaminants and their impacts can come from public data sources and/or private resources 1602, such as consumer confidence reports, regulations, monitoring rules, etc. Each of the contaminants will reduce the overall water quality index score, with contaminants from Tier I 1610 reducing the overall score more than contaminants from Tier II 1608, etc., if the quantity of the contaminants were equal. If the quantity of a Tier II 1608 contaminant were sufficiently high, it could possibly reduce the Water Quality Index score 1614 more than a minute amount of a Tier I 1610 contaminant.

FIG. 17 illustrates an example of water quality indexing and associated recommendations. In this example, the system retrieves an address for a particular geographic location, executes a search of known information about the geographic location (such as watershed information, water levels, contaminants, databases, etc.), produces a report on the retrieved information along with a score indicating the overall water quality for that location. The system can also access a database of an online vendor, identify what products they have which could help raise the water index score, and suggest that product to the user. For example, if the report indicates the presence of a particular contaminant, and the vendor has a filter which can remove that contaminant, the system may recommend that particular filter to the user. The system can also transmit an event notification, notifying the user of particular circumstances or situations regarding their water quality. For example, if the system detects that extreme rainfall in the mountains has led to flooding and additional contaminants within the downstream water supply, the system can generate and transmit an event notification to a user associated with a downstream address or location.

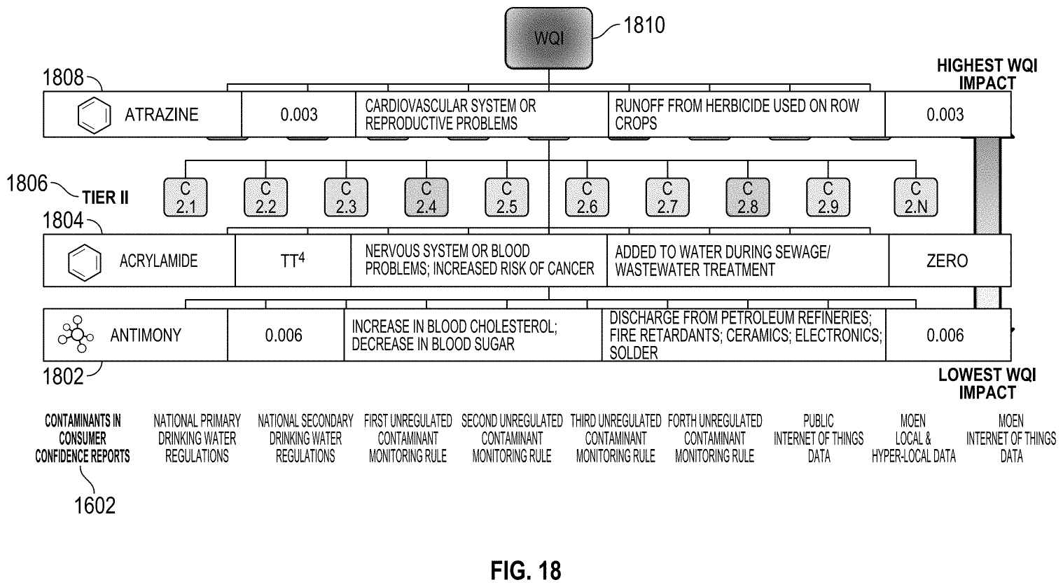

FIG. 18 illustrates a second example of water quality indexing based on tiered contaminants. In this example, various contaminants, having various levels of impact on human health, are detected in the water. For example, Atrazine 1808 is detected, which can have a high impact, as well as Antimony 1802, which has a relatively lower impact. Trace amounts of Acrylamide 1804 are detected, and because the amounts fall beneath established thresholds for harm, no impact from the Acrylamide 1804 is identified. Like the example of FIG. 16, the sources for the data 1602 which result in tiers can be public reports, databases, and/or other sources.

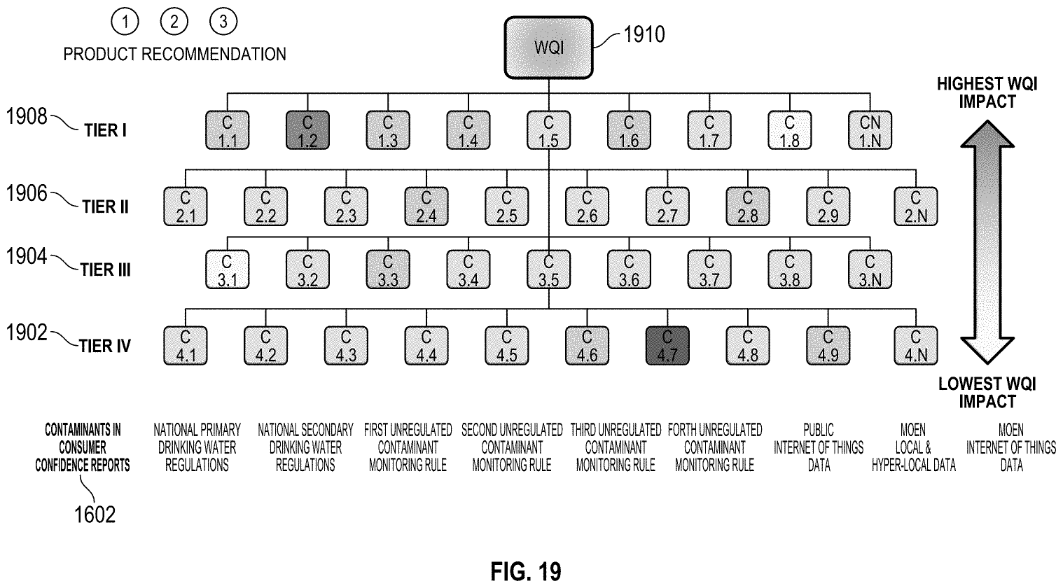

FIG. 19 illustrates a third example of water quality indexing based on tiered contaminants 1902, 1904, 1906, 1908, with the respective impacts of the detected chemicals illustrated. For example, a contaminant with high impact (based on both the amount detected as well as its tiered classification) is illustrated as darker, whereas a contaminant with relatively low impact is illustrated as having less shading. Again, the data used for the tiered classifications can come from public sources 1602.

The Water Quality Index score can, for example, be on a 0-100 spectrum, with “70” representing a passing score. In other configurations the Water Quality Index score can be a letter grade, such as “A” to “F”. The MCGL (Maximum Contaminant Level Goal) for a particular location can be provided to users, and can vary from location to location as required for the particular filtration systems available at that location and the water usage. For example, the MCGL may differ between agricultural use and drinking water or other uses, based on the respective health and/or environmental impact of the contaminants on the particular use in question.

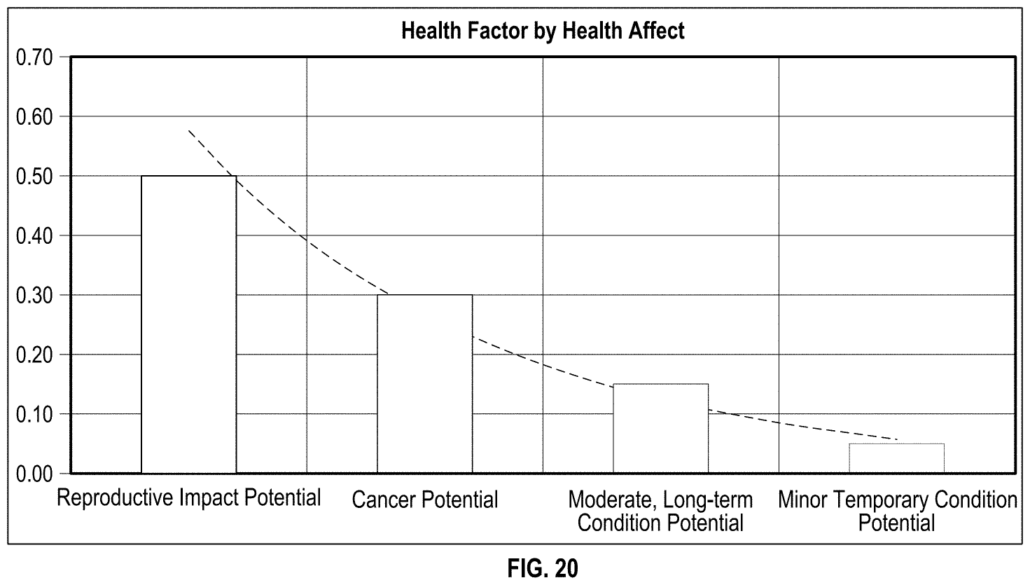

FIG. 20 illustrates an example of ranking health factors, which can be used in identifying in which tier various contaminants should be placed. As illustrated, contaminants with reproductive impact rank highest in health affect, followed by contaminants with cancer potential, then moderate/long-term health conditions, and finally minor/temporary conditions. In other circumstances or configurations, these respective rankings may change.

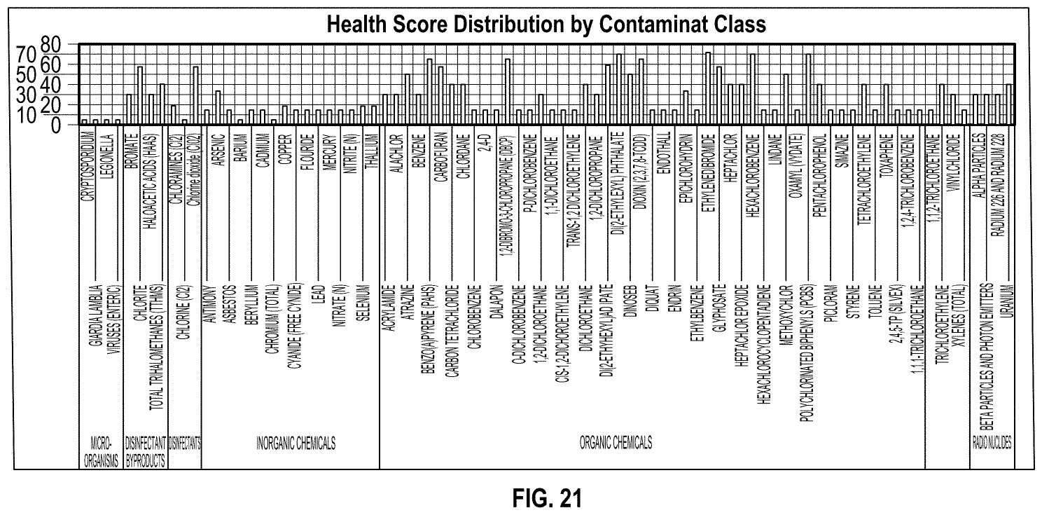

FIG. 21 illustrates an example of clustering contaminants by type. In this example, the respective contaminants are organized into the type of class. Examples illustrated can include inorganic chemicals, organic chemicals, micro-organisms, disinfectants, disinfectant byproducts, radionuclides. In other configurations, more or less categories/types of contaminants can be present. The impact of the contaminants on the Water Quality Index score can be based on the type of contaminant to which the contaminant belongs.

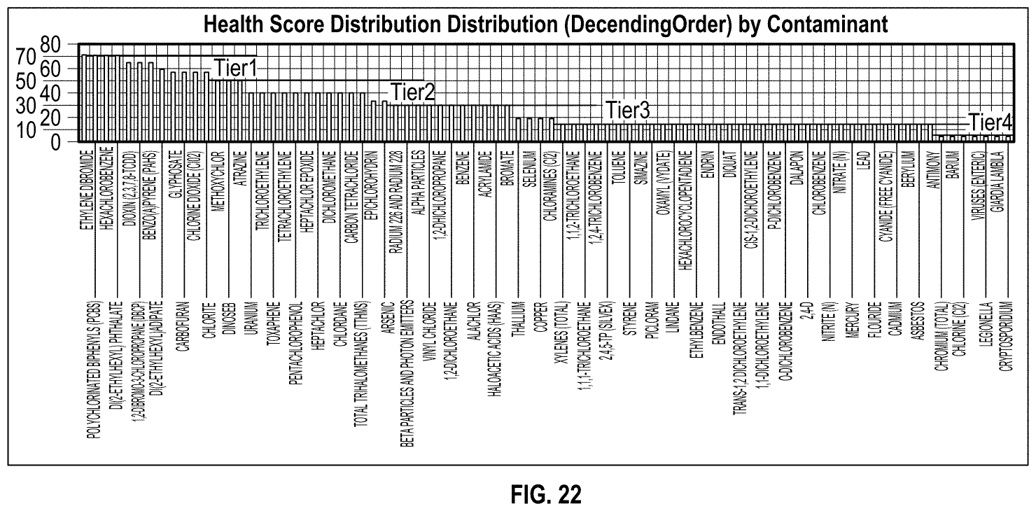

FIG. 22 illustrates an example of ranking contaminants. In this example, the contaminants are organized by the health impact each respective contaminant may have, allowing the contaminants to be organized into the respective tiers described above. The impact of the contaminants on the water quality index score can be based on the tier two which the contaminants are assigned.

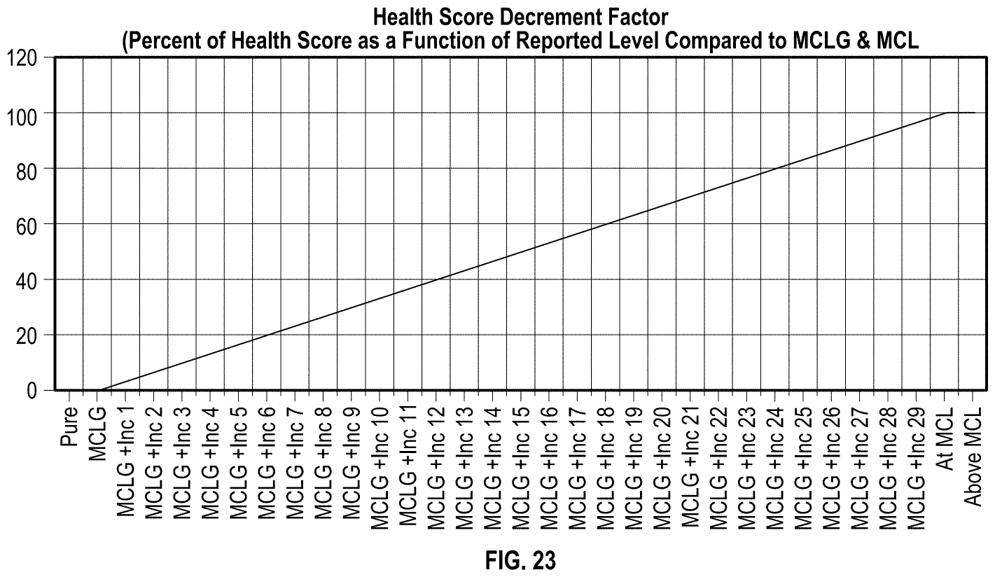

FIG. 23 illustrates an example of scoring water quality based on contaminant scores, compared to the Maximum Contaminant Level Goal (MCLG) and/or Maximum Contaminant Level (MCL). As illustrated, the goal in this example is for the MCLG to be at 0, or equivalent to pure water, whereas the MCL is set to be 100, or maximum contaminants. Each respective contaminant increases the overall decrement factor, effectively reducing the water quality index score. As illustrated, the decrement factor increases linearly across the various contaminants. However, in many instances the decrement factor will change in a nonlinear manner depending on the type of contaminant detected, the quantity of that contaminant, etc.

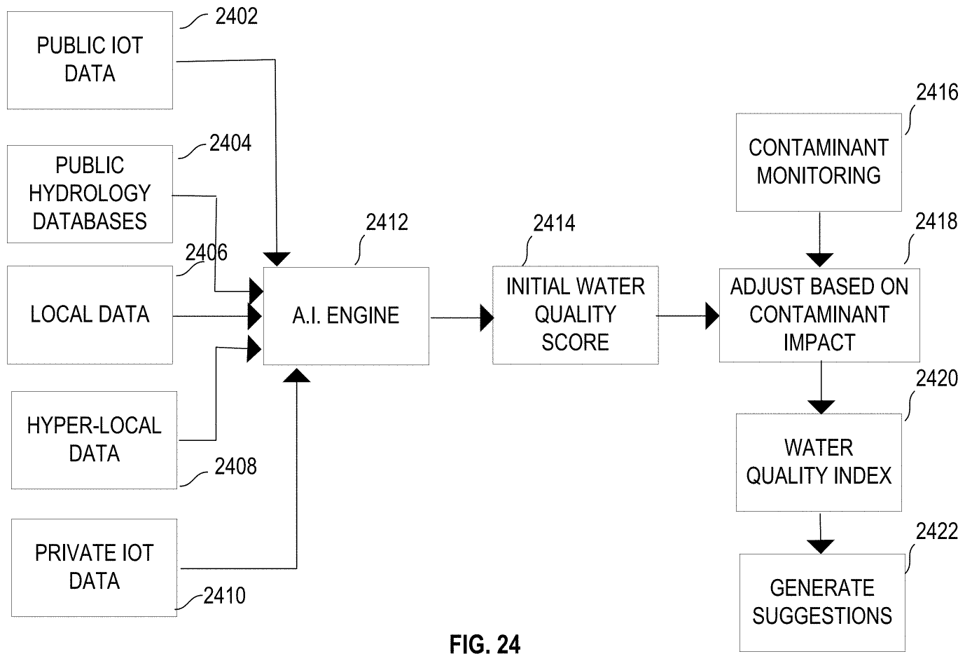

FIG. 24 illustrates an example of using an A.I. engine 2412 in sequence with contaminant monitoring to create a water quality index 2420 and associated suggestions 2422. As illustrated, various types of data (such as public IOT data 2402, public hydrology databases 2404, local data 2406, hyper-local data 2408 (such as data associated with a precise GPS location), and/or private IOT data 2410) are received, then input into an A.I. engine 2412. The A.I. engine 2412 then outputs an initial water quality score 2414, which can be adjusted 2418 based on contaminant impact, where the contaminants are monitored 2416 using various contaminant sensors. The system then outputs the adjusted water quality score as a water quality index 2420, and can also generate suggestions 2422 for how the user can improve or otherwise adjust the water quality index score 2420. While illustrated as distinct steps, the impact of contaminants 2416 on the overall score 22420 can be part of the A.I. engine 2412, such that generation of an intermediate score 2414, then adjusting that score 2418, takes place as part of the overall A.I. engine 2412. In yet other configurations, there may not be an intermediate score 2414, such that the contaminants 2416 are used in producing the initial water quality index score 2414.

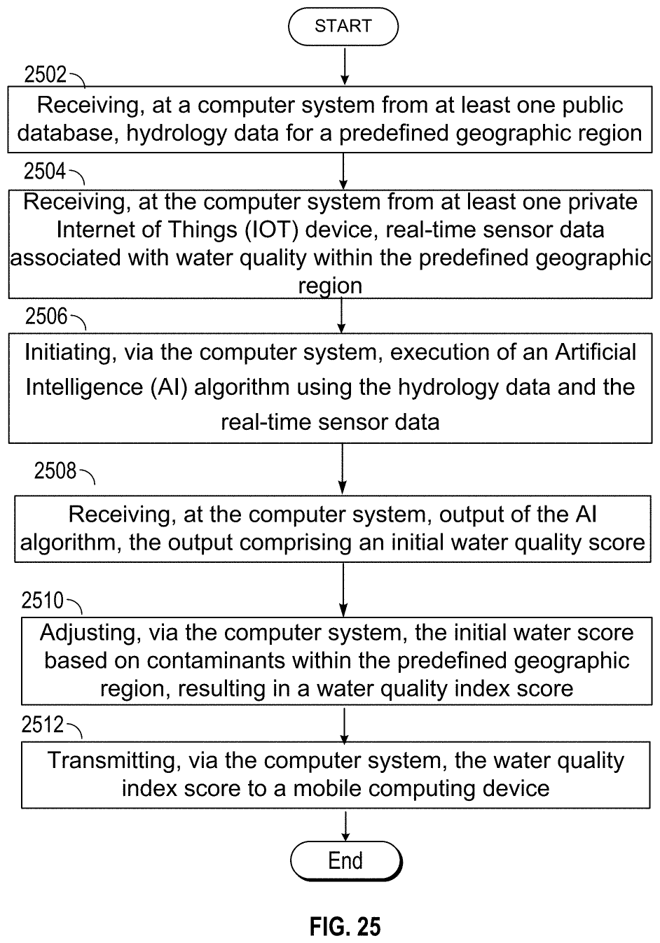

FIG. 25 illustrates an example method embodiment. The intent behind the illustrated method is that the water quality index score be effectively used and understood by persons responsible for water quality, health, and safety in the government, in the private sector, as well as at a consumer level, thereby preventing hazardous water contaminant related incidents and enabling the most efficient use of human and financial resources in an environmentally and socially responsible manner.

In this example, a computer system configured as disclosed herein receives, from at least one public database, hydrology data for a predefined geographic region (2502). The system receives, from at least one private Internet of Things (IOT) device, real-time sensor data associated with water quality within the predefined geographic region (2504) and initiates execution of a serverless Artificial Intelligence (AI) algorithm using the hydrology data and the real-time sensor data (2506). The system receives output of the serverless AI algorithm, the output comprising an initial water quality score (2508), and adjusts the initial water score based on contaminants within the predefined geographic region, resulting in a water quality index score (2510). The system then transmits the water quality index score to a mobile computing device (2512).

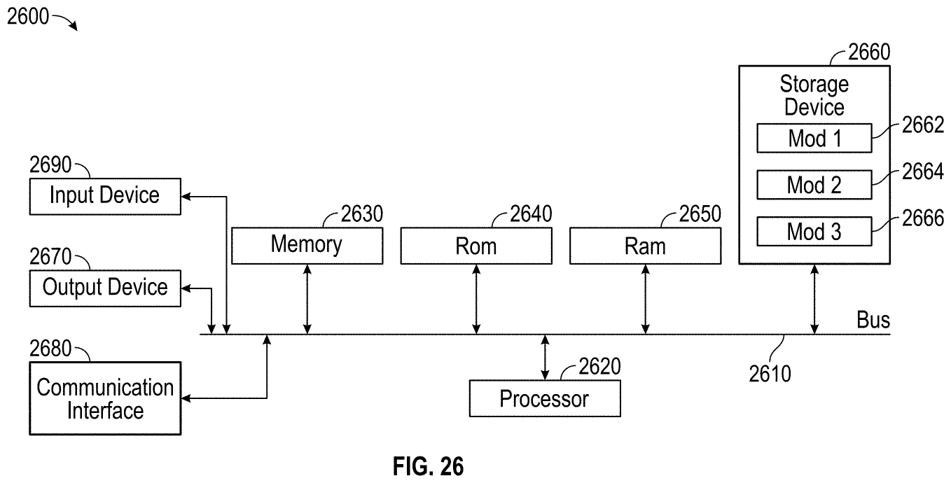

With reference to FIG. 26, an exemplary system includes a general-purpose computing device 2600, including a processing unit (CPU or processor) 2620 and a system bus 2610 that couples various system components including the system memory 2630 such as read-only memory (ROM) 2640 and random access memory (RAM) 2650 to the processor 2620. The system 2600 can include a cache of high-speed memory connected directly with, in close proximity to, or integrated as part of the processor 2620. The system 2600 copies data from the memory 2630 and/or the storage device 2660 to the cache for quick access by the processor 2620. In this way, the cache provides a performance boost that avoids processor 2620 delays while waiting for data. These and other modules can control or be configured to control the processor 2620 to perform various actions. Other system memory 2630 may be available for use as well. The memory 2630 can include multiple different types of memory with different performance characteristics. It can be appreciated that the disclosure may operate on a computing device 2600 with more than one processor 2620 or on a group or duster of computing devices networked together to provide greater processing capability. The processor 2620 can include any general purpose processor and a hardware module or software module, such as module 1 2662, module 2 2664, and module 3 2666 stored in storage device 2660, configured to control the processor 2620 as well as a special-purpose processor where software instructions are incorporated into the actual processor design. The processor 2620 may essentially be a completely self-contained computing system, containing multiple cores or processors, a bus, memory controller, cache, etc. A multi-core processor may be symmetric or asymmetric.

The system bus 2610 may be any of several types of bus structures including a memory bus or memory controller, a peripheral bus, and a local bus using any of a variety of bus architectures. A basic input/output (BIOS) stored in ROM 2640 or the like, may provide the basic routine that helps to transfer information between elements within the computing device 2600, such as during start-up. The computing device 2600 further includes storage devices 2660 such as a hard disk drive, a magnetic disk drive, an optical disk drive, tape drive or the like. The storage device 2660 can include software modules 2662, 2664, 2666 for controlling the processor 2620. Other hardware or software modules are contemplated, The storage device 2660 is connected to the system bus 2610 by a drive interface. The drives and the associated computer-readable storage media provide nonvolatile storage of computer-readable instructions, data structures, program modules and other data for the computing device 2600. In one aspect, a hardware module that performs a particular function includes the software component stored in a tangible computer-readable storage medium in connection with the necessary hardware components, such as the processor 2620, bus 2610, display 2670, and so forth, to carry out the function. In another aspect, the system can use a processor and computer-readable storage medium to store instructions which, when executed by the processor, cause the processor to perform a method or other specific actions. The basic components and appropriate variations are contemplated depending on the type of device, such as whether the device 2600 is a small, handheld computing device, a desktop computer, or a computer server.

Although the exemplary embodiment described herein employs the hard disk 2660, other types of computer-readable media which can store data that are accessible by a computer, such as magnetic cassettes, flash memory cards, digital versatile disks, cartridges, random access memories (RAMs) 2650, and read-only memory (ROM) 2640, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 2600, an input device 2690 represents any number of input mechanisms, such as a microphone for speech, a touch-sensitive screen for gesture or graphical input, keyboard, mouse, motion input, speech and so forth. An output device 2670 can also be one or more of a number of output mechanisms known to those of skill in the art. In some instances, multimodal systems enable a user to provide multiple types of input to communicate with the computing device 2600. The communications interface 2680 generally governs and manages the user input and system output. There is no restriction on operating on any particular hardware arrangement and therefore the basic features here may easily be substituted for improved hardware or firmware arrangements as they are developed.

Use of language such as “at least one of X, Y, and Z,” “at least one of X, Y, or Z,” “at least one or more of X, Y, and Z,” “at least one or more of X, Y, or Z,” “at least one or more of Y, and/or Z,” or “at least one of X, Y, and/or Z,” are intended to be inclusive of both a single item (e.g., just X, or just Y, or just Z) and multiple items (e.g, {X and Y}, {X and Z}, {Y and Z}, or {X, Y, and Z}). The phrase “at least one of” and similar phrases are not intended to convey a requirement that each possible item must be present, although each possible item may be present.

The various embodiments described above are provided by way of illustration only and should not be construed to limit the scope of the disclosure, Various modifications and changes may be made to the principles described herein without following the example embodiments and applications illustrated and described herein, and without departing from the spirit and scope of the disclosure,

We claim:

1. A method comprising:
receiving, at a computer system from at least one public database, hydrology data for a predefined geographic region;
receiving, at the computer system from at least one private Internet of Things (IOT) device, real-time sensor data associated with water quality within the predefined geographic region;
initiating, via the computer system, execution of a Artificial Intelligence (AI) algorithm using the hydrology data and the real-time sensor data;
receiving, at the computer system, output of the AI algorithm, the output comprising an initial water quality score;
adjusting, via the computer system, the initial water quality score based on contaminants within the predefined geographic region, resulting in a water quality index score; and
transmitting, via the computer system, the water quality index score to a mobile computing device.

2. The method of claim 1, wherein:
the AI algorithm receives as inputs:
the hydrology data;
the real-time sensor data;
weather data for the predefined geographic region;
a list of contaminants respectively associated with locations within the predefined geographic region; and
a period of time for which a predicted water quality score is desired;
the AI algorithm outputs:
the initial water quality score for the period of time; and
the AI algorithm:
uses the hydrology data, the real-time sensor data, and the weather data to predict water levels within the predefined geographic region, resulting in predicted water levels for the period of time;
uses the predicted water levels, the hydrology data, and the list of contaminants to predict a predicted spread of contaminants for the period of time; and
uses the predicted spread of contaminants and the real-time sensor data to predict future water quality for the period of time, resulting in the initial water quality score.

3. The method of claim 1, wherein the contaminants within the predefined geographic region comprise wastewater.

4. The method of claim 1, wherein the mobile computing device belongs to a civilian consumer.

5. The method of claim 1, wherein the AI algorithm uses solubility and weight of the contaminants to predict contaminant diffusion within the predefined geographic region.

6. The method of claim 1, further comprising:
detecting, via a contaminant sensor, a discrepancy between a predicted contaminant amount and an actual contaminant amount; and
modifying the AI algorithm based on the discrepancy.

7. The method of claim 6, wherein the modifying of the AI algorithm comprises replacing, in memory, at least one piece of data which resulted in the actual contaminant amount.

8. A system comprising:
at least one processor; and
a non-transitory computer-readable storage medium having instructions stored which, when executed by the at least one processor, cause the at least one processor to perform operations comprising:
receiving, from at least one public database, hydrology data for a predefined geographic region;
receiving, from at least one private Internet of Things (IOT) device, real-time sensor data associated with water quality within the predefined geographic region;
initiating execution of a Artificial Intelligence (AI) algorithm using the hydrology data and the real-time sensor data;
receiving output of the AI algorithm, the output comprising an initial water quality score;
adjusting the initial quality water score based on contaminants within the predefined geographic region, resulting in a water quality index score; and
transmitting the water quality index score to a mobile computing device.

9. The system of claim 8, wherein:
the AI algorithm receives as inputs:
the hydrology data;
the real-time sensor data;
weather data for the predefined geographic region;
a list of contaminants respectively associated with locations within the predefined geographic region; and
a period of time for which a predicted water quality score is desired;
the AI algorithm outputs:
the initial water quality score for the period of time; and
the AI algorithm:
uses the hydrology data, the real-time sensor data, and the weather data to predict water levels within the predefined geographic region, resulting in predicted water levels for the period of time;
uses the predicted water levels, the hydrology data, and the list of contaminants to predict a predicted spread of contaminants for the period of time; and
uses the predicted spread of contaminants and the real-time sensor data to predict future water quality for the period of time, resulting in the initial water quality score.

10. The system of claim 8, wherein the contaminants within the predefined geographic region comprise wastewater.

11. The system of claim 8, wherein the mobile computing device belongs to a civilian consumer.

12. The system of claim 8, wherein the AI algorithm uses solubility and weight of the contaminants to predict contaminant diffusion within the predefined geographic region.

13. The system of claim 8, the non-transitory computer-readable storage medium having additional instructions stored which, when executed by the at least one processor, cause the at least one processor to perform operations comprising:
detecting, via a contaminant sensor, a discrepancy between a predicted contaminant amount and an actual contaminant amount; and
modifying the AI algorithm based on the discrepancy.

14. The system of claim 13, wherein the modifying of the AI algorithm comprises replacing, in memory, at least one piece of data which resulted in the actual contaminant amount.

15. A non-transitory computer-readable storage medium having instructions stored which, when executed by at least one processor, cause at least one processor to perform operations comprising:
receiving, from at least one public database, hydrology data for a predefined geographic region;
receiving, from at least one private Internet of Things (IOT) device, real-time sensor data associated with water quality within the predefined geographic region;
initiating execution of a Artificial Intelligence (AI) algorithm using the hydrology data and the real-time sensor data;
receiving output of the AI algorithm, the output comprising an initial water quality score;
adjusting the initial water quality score based on contaminants within the predefined geographic region, resulting in a water quality index score; and
transmitting the water quality index score to a mobile computing device.

16. The non-transitory computer-readable storage medium of claim 15, wherein:
the AI algorithm receives as inputs:
the hydrology data;
the real-time sensor data;
weather data for the predefined geographic region;
a list of contaminants respectively associated with locations within the predefined geographic region; and
a period of time for which a predicted water quality score is desired;
the AI algorithm outputs:
the initial water quality score for the period of time; and
the AI algorithm:
uses the hydrology data, the real-time sensor data, and the weather data to predict water levels within the predefined geographic region, resulting in predicted water levels for the period of time;
uses the predicted water levels, the hydrology data, and the list of contaminants to predict a predicted spread of contaminants for the period of time; and
uses the predicted spread of contaminants and the real-time sensor data to predict future water quality for the period of time, resulting in the initial water quality score.

17. The non-transitory computer-readable storage medium of claim 15, wherein the contaminants within the predefined geographic region comprise wastewater.

18. The non-transitory computer-readable storage medium of claim 15, wherein the mobile computing device belongs to a civilian consumer.

19. The non-transitory computer-readable storage medium of claim 15, wherein the AI algorithm uses solubility and weight of the contaminants to predict contaminant diffusion within the predefined geographic region.

20. The non-transitory computer-readable storage medium of claim 15, having additional instructions stored which, when executed by the at least one processor, cause the at least one processor to perform operations comprising:
detecting, via a contaminant sensor, a discrepancy between a predicted contaminant amount and an actual contaminant amount; and
modifying the AI algorithm based on the discrepancy.
